(19) United States
(12) Plant Patent Application Publication
(10) Pub. No.: US 2004/0025214 P1
(43) Pub. Date: Feb. 5, 2004

(54) Hibiscus plant named: 'Cherub'

(76) Inventors: Gretchen Angela Zwetzig, Lincoln, NE (US); David Whitman Fleming, Lincoln, NE (US)

Correspondence Address: Gretchen Zwetzig, 8101 S. 14th St., Lincoln, NE, 68512, US

(21) Appl. No.: 10/207,249

(22) Filed: Jul. 30, 2002

(51) Int. Cl. A01H 5/00

(52) U.S. Cl. PLT/257

(57) ABSTRACT

A new and distinct cultivar of winter hardy hibiscus plant named ‘Cherub’ is the result of a unique hybridization. This new and distinct cultivar is characterized primarily as to novelty by its tropical aspects, its extreme cold hardiness to Zone 4, its medium-sized, thick-textured, tubular pink flowers with recurved petal edges and its shiny “Maple-like” green leaves and unusual pyramidal compactness of about 3 feet, its vigorous and uniform growth habit, and its floriferous nature from midsummer until frost, and its genetic achievement as a cross between a tropical and hardy Hibiscus.

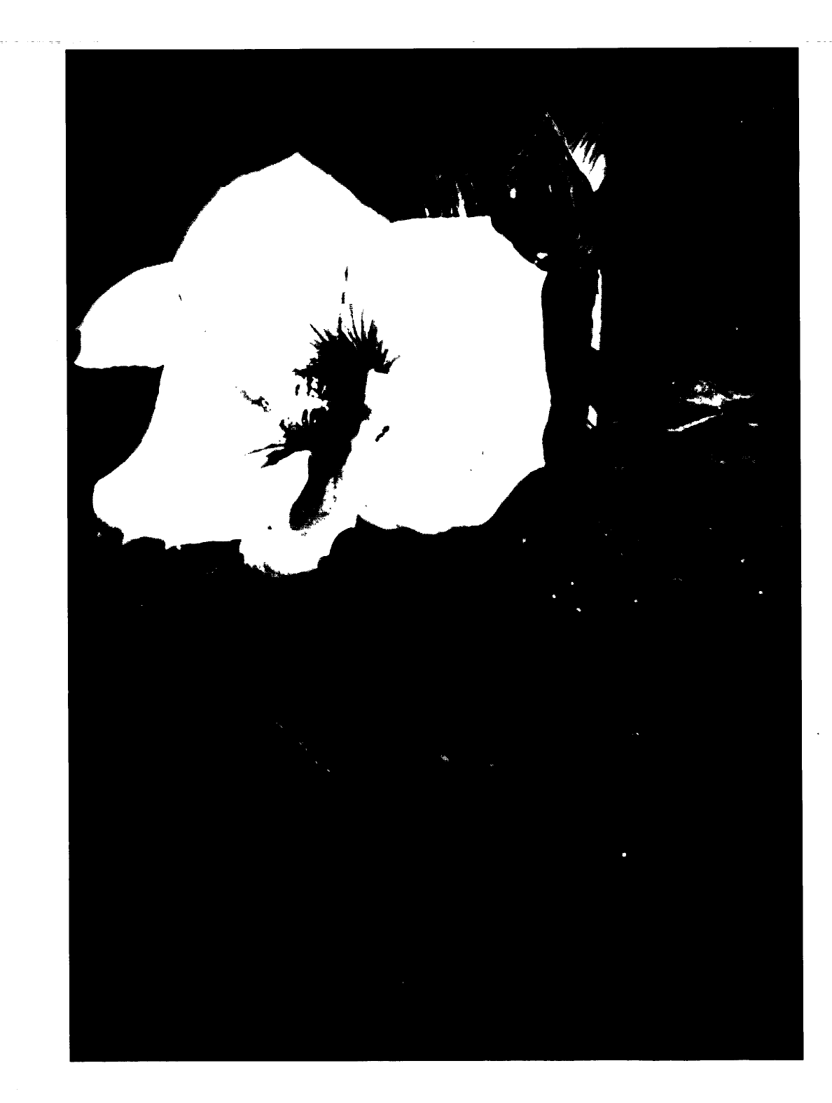

### BACKGROUND OF THE NEW PLANT

The new plant of this invention is the result of a unique hybridization, with the breeding achievement being evidenced in the outstanding combination of characteristics exhibited by this new and distinct Hibiscus plant, which include:

(a) Refined and noticeably shiny, “Maple-like”, green leaves which have smooth margins and pink buds which combined compliments the landscape;

(b) Medium-sized flowers that are outstanding for their soft pink overlapping petals with tubular shape having recurved petal edges;

(c) The plant being very floriferous with flowers that stay open at least one full day, sometimes two;

(d) The plant being small but vigorous with stouter stems, compact and “pyramidal” breaking action which gives it unique landscape utility; and

(e) The plant being so hardy that it can consistently withstand winter temperatures of at least −30 degrees Fahrenheit.

(f) The plant being a genetic achievement as a hybrid between a tropical and hardy Hibiscus.

### SUMMARY OF THE INVENTION

‘Cherub’ was the result of a 40-year breeding program. Its ancestry includes Hibiscus moscheutos and Hibiscus rosa-sinensis. More specifically, the plant resulted from multiple crossings with an unnamed Fleming Hibiscus rosa-sinensis (nonpatented) and an unnamed Fleming Hibiscus moscheutos (nonpatented). The seedling which most nearly met all of the above named standards was selected.

This new plant first bloomed in the summer if 1990 and was selected by David Fleming and Gretchen Zwetzig on David Fleming's property in Lincoln, Nebr. Asexual propagation of the plant by cuttings and root division has shown that the unique and distinguishing features of the plant are faithfully transmitted from generation to generation and appear to be fixed.

Since its origin, the plant has bloomed from midsummer until frost, while exhibiting the aforementioned distinctive characteristics. This Tropical-Hardy™ Hibiscus plant greatly contributes to the market with its tropical aspect, sheer beauty and utter refinement, its compact growth habit, its enhanced resistance to disease and insects, its stability through extremes in rain and drought, and its extreme hardiness.

### BRIEF DESCRIPTION OF THE DRAWINGS

The flower and foliage of the cultivar are shown in the attached photographs. More specifically, Photograph 1 is a close-up of the flower and the leaf.

The colors are as true as is reasonably possible to attain in photographic illustrations of this type. The colors illustrated may be slightly off due to light reflectance.

### DESCRIPTION OF THE NEW PLANT

What follows is a detailed description of the new cultivar. The specific color descriptions are in accordance with the Dictionary of Color (Maerz and Paul), while general color recitations are consistent with ordinary American color terminology.

‘Cherub’ has not been observed under all possible environment conditions. It is to be understood that the phenotype may vary significantly with variations in environment such as temperature, light intensity, and day length, without, however any difference in genotype of the plant. The following botanical characteristics and observations are gathered from the plant when grown in Lincoln, Nebr. (USDA Zone 4).

### THE PLANT

Parentage:

Seed parent.—Hibiscus moscheutos, Unnamed and unpatented Fleming seedling.

Pollen Parent.—Hibiscus rosa-sinensis Unnamed and unpatented Fleming seedling.

Origin.—A selected seedling from stated cross.

Type of plant.—Hardy, herbaceous perennial with some tropical genetics.

Classification.—Variety of Tropical-Hardy™ Hibiscus plant.

Propagation.—Relatively easy to propagate; holds its distinguishing characteristics through successive cuttings and division.

Size.—About 3 feet tall and about 3 ft. wide.

Form.—Symmetrical annual growth from perennial roots.

Habit.—Upright and uniformly branched.

Growth.—Vigorous; small and very compact. Full grown (3 ft.) in 2 years.

Hardiness.—Hardy to at least −30 degrees Fahrenheit.

General health.—Plant is very disease resistant against rot, aphids, spider mites, scale, white fly, etc.; sturdy through excessive drought, rain, or wind.

Propagation.—Relatively easy and fast rooting in approximately 2 weeks.

Foliage:

1. Arrangement.—Alternate.

2. Shape.—Juvenile: Variable, 3-lobed leaf. Rounded base shape. Mature: Refined, “maple-like”, 3-5 lobed leaf with entire to extremely mildly serrate margins and acute apices; rounded base.

3. Size.—Mature leaf (petiole base to apex): Length is about 4 inches or 11 cm, width is about 3½ inches or 8½ cm.

4. Color.—Young leaves (upper side): L-5, PLATE 23, page 69. Mature leaves: (upper side): J-2, PLATE 24, page 71. Veins: L-3, PLATE 23, page 69. Coloration of the lower leaf surface: L-5, PLATE 23, page 69.

5. Surface texture.—Medium to High sheen.

6. Petiole length.—About 1¼ inches or 3½ cm.

7. Petiole color.—Green to K-1, PLATE46, page 115.

Stems: The stems are stout at the base, becoming more slender toward the apices; coloration is consistent with the petiole.

### THE FLOWER

Blooming period: Profuse from July until frost.

Flower diameter: Medium to large; about 6½ inches (16 cm).

Borne: Singly in leaf axils, from midpoint of stems upward.

Bloom duration: Individual bloom lasts at least one full day, sometimes 2.

Form: Campanulate; petals are also very overlapping with a conical shape and recurved petal edges.

Bud:

1. Length.—About 2¾ inches (or 7 cm) on day before opening.

2. Color.—J-5, PLATE 4, page 31.

3. Surface texture.—Semi-glabrous.

4. Duration.—One to two weeks.

Corolla: Having 5 petals.

1. Petal character and dimensions.—Campanulate and very overlapping. Length is about 4¼ inches or 11 cm. Width is about 4¼ inches or 11 cm.

2. Coloration.—Pale pink petals. (a) Pale pink petals: F-2, p. 29, PLATE 3. Coloration of lower petal surface: Slightly lighter than F-2, pg. 29, PLATE 3. (b) Veination in petals: 1-3, pg. 29, PLATE 3. (c) Eye Zone: L-5, pg. 29, PLATE 3.

3. Texture.—Smooth.

4. Substance.—Thick.

5. Shape.—Campanulate and very overlapping; tubular with recurved petal edges.

Reproductive organs:

1. Staminal column.—Stamen along basal two-thirds. Stamen length: About 2¼ inches (or 5.5 cm). Stamen color: B-1, pg. 29, PLATE 2. Pollen color: E-2, pg 31, PLATE 4.

2. Style.—Terminates upper one-third of staminal coumn. Stigmatic lobe color: A-6, pg. 45, PLATE 11.

Calyx: Connate at the base; about 1¾ inches (or 4.5 cm) in length.

Color.—Medium green.

Stipules: None.

Epicalyx: Whorl of bracts.

1. Shape.—10 to 12 slender, sword-shaped bractlets.

2. Length.—About 1 inches (or 2¼ cm).

3. Color.—L-5, pg 67, PLATE 22.

Penduncle:

1. Length.—About 2½ inches (or 6.5 cm).

2. Strength.—stiff and sturdy.

3. Color.—Light to medium green.

### GENERAL OBSERVATIONS

This plant, as a hybridized Tropical-Hardy™ Hibiscus, is valuable to the landscape market for its improvements in tropical aspect, pink tubular flowers with recurved edges, shiny and very refined, “Maple-like” green foliage on a much more compact, “pyramidal” plant, its all-around vigor, good breaking action, and adaptation to extreme environments, including the ability to consistently survive winter temperatures of at least −30 degrees Fahrenheit.

1. A new and distinct cultivar of Tropical-Hardy™ Hibiscus plant, as herein shown and described, characterized by its tropical aspect, extremely compact, “pyramidal” nature, shiny, very refined green, “Maple-like” foliage, its pale pink tubular flowers with recurved petal edges, its vigor, and its hardiness.
